(19) United States
(12) Patent Application Publication
(10) Pub. No.: US 2019/0029630 A1
(43) Pub. Date: Jan. 31, 2019

(54) MEDICAL INSTRUMENT AND METHOD FOR OPERATING SUCH A MEDICAL INSTRUMENT FROM A REMOTE SITE

(71) Applicant: Siemens Healthcare GmbH, Erlangen (DE)

(72) Inventors: Katharina Aschoff, Erlangen (DE); Dieter Faust, Erlangen (DE); Maria Kroell, Erlangen (DE); Stephan Nufer, Erlangen (DE)

(73) Assignee: Siemens Healthcare GmbH, Erlangen (DE)

(21) Appl. No.: 16/045,990

(22) Filed: Jul. 26, 2018

(30) Foreign Application Priority Data
Jul. 26, 2017 (EP) 17183355

(51) Int. Cl. A61B 6/00; A61B 5/055; G16H 40/63; G16H 40/67

(52) U.S. Cl. CPC A61B 6/548; A61B 5/055; G16H 40/67; A61B 6/563; G16H 40/63; A61B 6/461

(57) ABSTRACT

In a method for using a medical instrument, in particular a medical imaging device, the medical instrument is selectively operable in a first mode giving access to the medical instrument and in a second mode giving limited access, in particular refusing access, to the medical instrument. When access to the medical instrument is requested, by using a human-machine-interface of the medical instrument, i.e. in an attended mode, the medical instrument is transferred into the first mode or into the second mode in response to the request. The medical instrument is also transferred into the first mode by using a second human-machine interface at a remote site, when the first human-machine-interface of the medical instrument is inactive for a defined period of time, in particular after requesting the access, i.e. in an unattended mode.

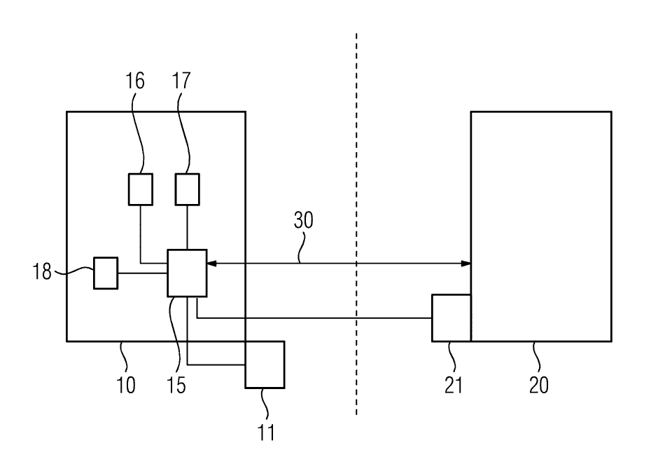

### BACKGROUND OF THE INVENTION

### Field of the Invention

The present invention concerns a method for using a medical instrument, a system that includes a medical instrument and a remote access device, and a non-transitory computer readable data storage medium that implements such a method.

### Description of the Prior Art

Medical imaging instruments, such as a magnetic resonance tomography (MRT) scanner, an X-ray scanner, a computer tomography (CT) scanner or similar devices, are well known. Typically, a first operator is present at the medical instrument, who operates the medical instrument using a human-machine-interface of the medical instrument. For example, the first operator can start and finish a recording of a medial image, can configure the settings of the medical imaging device and/or can protocol the recorded images. For giving a second operator the chance to operate the medical imaging device from a remote site, the first operator has to choose between refusing access of the second operator to the medical instrument or selecting the extent of the access intended for the second operator. As a result, the first operator needs to be available at the human-machine-interface of the medical instrument, when the second operator wants to access the medical instrument.

On a very general level, remote service aspects in connection with medical imaging devices are disclosed in DE 102012203975 A1, DE 102012202362 A1, DE102012205296 A1 and DE 102014220808 B4, for example.

### SUMMARY OF THE INVENTION

An object of the present invention is to provide a method for gaining access to a medical instrument from a remote site in an unattended state of the medical instrument.

According to a first aspect of the invention a method for using a medical instrument, in particular a medical imaging device, is provided, wherein the medical instrument is selectively operable in a first mode giving access to the medical instrument and in a second mode giving limited access, in particular refusing access, to the medical instrument. When access to the medical instrument is requested, by using a human-machine-interface of the medical instrument, i.e. in an attended mode, the medical instrument is transferred into the first mode or into the second mode in response to the request. The medical instrument is also transferred into the first mode by using a second human-machine interface at a remote site, when the first human-machine-interface of the medical instrument is inactive for a defined period of time, in particular after requesting the access, i.e. in an unattended mode.

In contrast to the state of the art, the medical instrument is transferred into the first mode via the remote site, when the human-machine-interface is inactive, in particular for the defined period of time. As a consequence, a second operator can access the medical instrument via the human-machine-interface at the remote site, even when the first operator does not interact with the first human-machine interface, for example because the first human-machine-interface of the medical instrument is unattended. Preferably, the medical instrument is a medical imaging device, such as a magnetic resonance tomography (MRT) scanner, an X-ray scanner, a computer tomography (CT) scanner or a similar device. For instance, operating such a medical instrument means configuring a setting of the medical instrument, starting or finishing a recording of a medical imaging, protocolling the medical imaging and administering the medical imagines in a memory device, in particular by using the first human-machine-interface of the medical instrument or the second human-machine-interface of the remote site. Either human-machine-interface can be a touchscreen, a tablet, a smartphone, a keyboard or a console. Preferably, the remote site has full access in the first mode and as a consequence the second operator can operate the medical instrument, but the remote site has only access to a current image on a display of the medical instrument, the access is refused, in the second mode. Preferably, in the first mode and/or the second mode, specified functions, such as recording a medical imaging or moving a table of the medical instrument, are prevented or blocked. It is also conceivable that there are several second modes each having different extents of access to the medical instrument, and the medical instrument is transferred to one of the several second modes. The extent of access in the first mode is bigger than the extent of access in the second mode, or one of the several second modes.

The term “remote site” preferably has a computer or workstation located spatially apart from the medical instrument, for example in another room, another floor and/or another building. Preferably, the medical instrument and the remote site exchange data and/or commands via a communication channel (link), such as a wireless or a wired connection. Furthermore, the medical instrument has a control computer for operating the medical instrument, wherein the control computer includes a processor that configures a setting of the medical instrument, starting and finishing the recording of a medical imaging, protocolling the medical imaging and administering the medical imagines in a memory.

The term “inactive” describes a state, in which the human-interface-machine is not used and/or no selection between the first mode and the second mode has been chosen by using the human-machine interface during a defined period of time. The period of time is triggered or started by the request. For example, the period of time lasts between 0.5 minutes and 10 minutes, preferred 0.7 and 5 minutes and most preferred about 1 min. Such a request is initiated by the human-machine-interface at the remote site. Preferably, the first operator is informed about the request on a screen of the medical instrument immediately.

In a preferred embodiment, the method further includes the step of transferring the medical instrument into the first mode, when the first human-machine-interface is inactive for a defined period of time after requesting the access, and the medical instrument is in a non-use state. Thus, it is avoided that the remote site accidentally configures the settings of the medical instrument during operation, in particular during recording or configuring the settings for a patient in the unattended state. The medical instrument is considered to be in a use-state, for example, when a patient is logged in, and is considered to be in a non-use state, for example, when no patient is logged in. Preferably, the remote site is informed whether the medical instrument is in the use-state or the non-use state at the point of time of the request made at the remote site.

In a further embodiment of the present invention, the medical instrument is transferred into the first mode or into the second mode automatically, when the first human-machine-interface is inactive for a defined period of time before the request. Thus, the second operator can directly access the medical instrument, without waiting for expiration of the set period of time after requesting the time. In other words, the period of time in which the human-machine-interface is inactive already expired before the request. It is also possible to acquire that the defined period of time must expire before access is reduced in the second mode, when the medical instrument was inactive before the request.

Preferably, information about a remaining time in the first mode or the second mode is provided at the remote site. Thus, the second operator is informed about the time left for finishing or her task and as consequence he or she can organize the remaining work effectively. In particular, a countdown is provided informing the second operator. It is also possible to activate an alarm, for example an acoustical or visual alarm, at defined points of time during the countdown.

According to another embodiment of the present invention, the first mode and/or the second mode is terminated by the first human-machine-interface at the medical instrument or by the second human-machine-interface at the remote site. In particular, terminating the first mode and/or second mode by one of the human-machine-interfaces of the medical instrument allows observing the actions of the second operator and stopping the access of the second operator for safety reason. In the case that the first mode and/or the second mode is terminated by the second human-machine-interface at the remote site, it is preferably provided that the medical instrument, in particular the first operator at the medical instrument, is informed about the termination, and the first operator can resume his or her work.

Preferably, transferring the medical instrument into the first mode or into the second mode, in particular into one of several second modes, by using the second human-machine interface at the remote site, is selected by a management system. Thus, it is possible to select the first mode or the second mode depending on the respective type of the second operator. Therefore, the management system is configured such that the first mode, the second mode, and/or one of the several second modes, is assigned to the respective second operator. Thus, it is possible for the management system to assign the first mode, the second mode, and/or the individual second mode to the respective second operator individually. It is also possible for the management system to be configured so as to require the second operator to identify himself or herself by a password, and subsequently the management system determines the corresponding first mode, the second mode, and/or the one of the several second modes based on the password.

In another embodiment, the management system is incorporated into the medical instrument or into a server. By incorporating the management system into the medical instrument, the first operator, or an administrator of medical devices that include the medical instrument, can determinate the extent of access for the individual second operator, without the need of an additional component that forms the management system. Alternatively, it is also possible to incorporate the management system into a server, such as a network or a cloud. As a consequence, it is possible to establish a central assignment between the amount of access and the individual second operator, for example for a number of medical instruments.

Preferably, a bidirectional communication between the medical instrument and the remote site is established in the first mode, and a unidirectional communication is established in the second mode. Thus, the second operator cannot modify the medical instrument in the second mode. In particular, data corresponding to the content of the current screen of the medical instrument are transferred to the remote site in the second mode.

According to another embodiment, information about the present usage of the medical instrument is provided at the remote site. Thus, it is possible to inform the second operator about the status of the current recording process and/or the planned recordings, for example, and the second operator can estimate the time until the medical instrument is in a non-use state.

In a further embodiment, the first mode and/or the second mode, administrative tasks are performed. Administrative tasks are, for example, a maintenance procedure or a protocol maintenance.

The present invention also encompasses a system that includes a medical instrument of the type described above that is operable on-site and from a remote site, and that includes a computer system distributed between the medical instrument on-site and the remote site, the computer system being configured to operate the system in order to implement any or all embodiments of the method according to the invention, as described above.

The present invention also encompasses a non-transitory, computer-readable data storage medium encoded with programming instructions that, when the storage medium is distributively loaded into a computer system of a system having a medical instrument that is operable on-site and from a remote site, cause the computer system to operate the medical instrument in order to implement any or all embodiments of the method according to the invention, as described above.

### BRIEF DESCRIPTION OF THE DRAWINGS

The single FIGURE is a block diagram illustrating schematically the method for configuring a medical instrument according to a preferred embodiment of the present invention.

### DESCRIPTION OF THE PREFERRED EMBODIMENTS

The FIGURE is a block diagram illustrating schematically a method for using a medical instrument 10 according to a preferred embodiment of the present invention. For example, the medical instrument 10 is a medical imaging device such as a magnetic resonance tomography (MRT) scanner, an X-ray scanner, a computer tomography (CT) scanner, or a similar device. Such a medical instrument 10 has a control computer 15 for configuring the setting of the medical instrument and operating the medical instrument 10. Preferably, for operating the medical instrument 0 the control computer 15 has a processor configured to implement settings 16 of the medical instrument 10, and/or a clinical task 18 of the medical instrument 10 can be started, observed and finished, and/or data can be stored in a memory 17 and/or data can be manipulated. The term “data” encompasses an image data set and/or patient related information such as a name or an identification of the patient. Furthermore, the medical instrument 10 has a first human-machine-interface 11 connected on-site to the control computer 15 for communication therewith. Such a first human-machine-interface 11 can be a touchscreen, a tablet, a smartphone, a keyboard or a console. Via the first human-machine-interface, a first operator of the medical instrument 10 has direct, on-site access to the control computer 15 for operating the medical instrument 10.

Furthermore, the system shown in the FIGURE has a remote site 20. For a communication, i. e. an exchange of data sets and/or or commands, between the medical instrument 10 and the remote site 20 a communication channel 30 is provided. Such a communication channel 30 can be realized by a wireless connection or a hardwired connection. The remote site 20 is located spatially apart from the medical instrument 10, for example in another room or floor. For operating the medical instrument 10 by a second operator at the remote site 20, the remote site 20 has a second human-machine-interface 21.

A prerequisite for operating the medical instrument 10 via the remote site 20 is that access to the medical instrument 10, in particular to the control computer 15, is given to the remote site 20, i. e. the second human-machine-interface 21 at the remote site 20. The medical instrument 10 can be selectively operated in a first mode giving the remote site 20 access to the medical instrument 10, and in a second mode giving the remote site 20 limited access to the medical instrument 10. For example, the remote site 20 can exchange both data and commands with the medical instrument 10, in particular with the control computer 15, in the first mode, and can exchange only data in the second mode such as transferring image data sets corresponding to a current display of a screen of the medical instrument 10.

For getting access to the medical instrument 10 from the remote site 20, in particular the control computer 15, a request for accessing the medical instrument 10 is transmitted from the remote site 20 to the medical instrument 10. The medical instrument 10 is configured such that the medical instrument 10 is transferred into the first mode or into the second mode in response to a request using the human-machine-interface 11 at the medical instrument 10. In other words, the first operators determines whether the medical instrument 10 is to be operated in the first mode or the second mode. It is also possible for access to the medical instrument 10 to be refused by the first human-machine-interface 11 of the medical instrument 10. Thus, it is advantageously possible to control all access via the human-machine interface 11 of the medical instrument 10. As a consequence, the first operator being located near to the medical instrument 10 can control the access of the second operator at the remote site 20 for safety reasons, provided that the first person is present at the medical instrument 10 or near to the human-machine-interface 11 of the medical instrument 10.

Furthermore, the medical instrument 10 can be transferred selectively into the first mode by using the human-machine interface 21 at the remote site 20, when the first human-machine-interface 11 of the medical instrument 10 is inactive for a defined period of time after requesting the access. Thus, it is advantageously possible to get access to the medical instrument 10 at the remote site 20 even when the first operator is not present at the first human-machine-interface 11 of the medical instrument 10, i. e. when the medical instrument 10 is unattended.

Furthermore, the control computer 15 is configured such that the approval for transferring the medical instrument 10 to the first mode and/or the second mode is given only when both the medical instrument 10 is in a non-use state, and the human-machine-interface 11 of the medical instrument 10 is inactive for a defined period of time after requesting the access. Thus, it is avoided that settings are changed accidentally during a treatment of a patient. A non-use state can be identified by a login state of a patient, i. e. when no patient is logged in, the medical instrument 10 is in the non-use state.

Furthermore, the control computer 15 can serve as a management system.

Preferably, the management system is configured so as to define the extent of access intended for the remote site 20. For example, the second operator of a first type (access category) can choose between the first mode and the second mode, whereas a second operator of a second type can only select the second mode. Furthermore it also possible for several second modes to be provided, and for the second mode to be adapted individually to the second operator of the second type. This is based on an assignment between the respective second operator and the individually defined second mode in the management system, in particular in a memory of the management system. It is also possible for the management system to incorporate in a server, such as a network or a cloud.

Although modifications and changes may be suggested by those skilled in the art, it is the intention of the Applicant to embody within the patent warranted hereon all changes and modifications as reasonably and properly come within the scope of the Applicant's contribution to the art.

1. A method for operating a medical instrument, comprising:
configuring a medical instrument so as to be selectively operable in a first mode that gives access to the medical instrument via an on-site location which is the location of the medical instrument, and in a second mode that gives limited access to the medical instrument;
placing said medical instrument in said first mode when a request for access to the medical instrument is made via a first human-machine interface made on-site at said location of said medical instrument; and
transferring said medical instrument into said second mode when a request for access to said medical instrument is made via a further human-machine interface at a remote site, when said medical instrument is inactive for a predetermined period of time.

2. A method as claimed in claim 1 wherein said medical instrument is an imaging device.

3. A method as claimed in claim 1 wherein said limited access in said second mode is a refusal of access to said medical instrument.

4. A method as claimed in claim 1 comprising beginning said predetermined period of time after requesting said access via said further human-machine interface.

5. A method as claimed in claim 1 comprising transferring said medical instrument into said first mode when said human-machine interface on-site is inactive for said predetermined period of time, or wherein said medical instrument is in a non-use state.

6. A method as claimed in claim 1 comprising automatically transferring said medical instrument into said first mode or said second mode, when said human-machine interface of said medical instrument on-site is inactive for a further predetermined period of time before said request for access.

7. A method as claimed in claim 1 comprising, at a display screen at said remote site, providing information that designates a remaining time in which said medical instrument can remain in said first mode or said second mode.

8. A method as claimed in claim 1 comprising terminating at least one of said first mode or said second mode by said on-site human-machine interface or by said further human-machine interface at said remote site.

9. A method as claimed in claim 1 comprising transferring said medical instrument into either said first mode or said second mode using a management system human-machine interface that is a part of a management system computer in communication with said medical instrument.

10. A method as claimed in claim 1 comprising combining said management system computer into said medical instrument or into a server in communication with said medical instrument.

11. A method as claimed in claim 1 comprising establishing a bidirectional communication between said medical instrument and said remote site in said first mode, and establishing a unidirectional communication between said medical instrument and said remote site in said second mode.

12. A method as claimed in claim 1 comprising displaying, at a display screen at said remote site, information describing a current usage state of said medical instrument.

13. A method as claimed in claim 1 comprising allowing administrative tasks with regard to said medical instrument to be performed in at least one of said first mode and said second mode.

14. A medical system comprising:
a medical instrument;
said medical instrument being configured so as to be selectively operable in a first mode that gives access to the medical instrument via an on-site location which is the location of the medical instrument, and in a second mode that gives limited access to the medical instrument;
said medical instrument being configured to be placed in said first mode when a request for access to the medical instrument is made via a first human-machine interface made on-site at said location of said medical instrument; and
said medical instrument being configured to be transferred into said second mode when a request for access to said medical instrument is made via a further human-machine interface at a remote site, when said medical instrument is inactive for a predetermined period of time.

15. A non-transitory, computer-readable data storage medium encoded with programming instructions, said storage medium being loaded into a computer system of a medical system that comprises a medical instrument, said programming instructions causing said computer system to:
configure the medical instrument so as to be selectively operable in a first mode that gives access to the medical instrument via an on-site location which is the location of the medical instrument, and in a second mode that gives limited access to the medical instrument;
place said medical instrument in said first mode when a request for access to the medical instrument is made via a first human-machine interface made on-site at said location of said medical instrument; and
transfer said medical instrument into said second mode when a request for access to said medical instrument is made via a further human-machine interface at a remote site, when said medical instrument is inactive for a predetermined period of time.
